(19) United States
(12) Patent Application Publication
(10) Pub. No.: US 2026/0117307 A1
(43) Pub. Date: Apr. 30, 2026

(54) METHOD FOR IN SITU DETECTION OF LUNG CANCER MARKERS AND TUMOR HETEROGENEITY IN LIVE CIRCULATING MALIGNANT CELLS

(71) Applicant: WUHAN UNIVERSITY, Hubei (CN)

(72) Inventors: Sixue CHENG, Hubei (CN); Xianzheng ZHANG, Hubei (CN); Di HAN, Hubei (CN); Qiyang HUANG, Hubei (CN); Qingyu GAO, Hubei (CN); Xinru LIAO, Hubei (CN); Jinju LEI, Hubei (CN); Jingping YUAN, Hubei (CN)

(73) Assignee: WUHAN UNIVERSITY, Hubei (CN)

(21) Appl. No.: 19/019,375

(22) Filed: Jan. 13, 2025

(30) Foreign Application Priority Data
Oct. 31, 2024 (CN) 202411540953.7

(51) Int. Cl. C12Q 1/6886; C12Q 1/6841

(52) U.S. Cl. CPC C12Q 1/6886; C12Q 1/6841; C12Q 2600/158

(57) ABSTRACT

The present application is related to a method for in situ detection of multiple lung cancer-related markers in live circulating malignant cells, and tumor heterogeneity, including accurately detecting at least one lung cancer-related nucleic acid marker in live lung cancer cells by using a lung cancer-targeted nanoprobe loaded with a plurality of molecular beacons to reflect levels of different markers in the lung cancer cells from a single cell level and a heterogeneous state of a tumor of a specific patient. The nanoprobe is a nanoparticle self-assembled from a high polymer material, an electropositive protein, a functional polypeptide and/or a functional aptamer, and a molecular beacon of a lung cancer-related marker. The nanoprobe can be used to target different phenotype circulating malignant cells in whole blood, detect nucleic acid markers in living cells, and reflect levels of different markers in the malignant cells from a single cell level.

### CROSS-REFERENCE TO RELATED APPLICATION

This application claims the priority benefit of China application no. 202411540953.7 filed on Oct. 31, 2024. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Dec. 27, 2024, is named 152150_SEQUENCELISTING and is 15,366 bytes in size.

### BACKGROUND OF THE INVENTION

### Technical Field

The present invention relates to the field of biomedical technologies, and specifically, to a method for in situ detection of multiple lung cancer-related markers in live circulating malignant cells, and tumor heterogeneity.

### Background of Related Art

According to Global Cancer Statistics 2020, lung cancer is the second most commonly diagnosed cancer and remains the leading cause of cancer death, with an estimated 2.2 million new cases of cancer and 1.8 million deaths. The lung cancer constitutes a major threat to human health primarily because it is associated with metastasis and recurrence. In the early stage of lung cancer, due to a small tumor volume, the tumor has weak compression and stimulation effects on surrounding normal tissues, such that patients have insidious symptoms, and consequently cannot be treated in time. As a result, many patients are already at a middle or late stage when diagnosed with lung cancer and miss the best opportunity for surgery, significantly reducing the cure rate.

At present, there are three major methods for screening lung cancer, including imaging examinations, cytological or histological techniques, and biomarkers. Imaging examinations bring the risk of radiation exposure to patients and molecular information is unable to be obtained, causing difficulties in subsequent applications of personalized diagnosis and treatment. When the cytological or histological techniques are used, as a biopsy is often required, which is an invasive procedure, sampling is often difficult to obtain from critical sites and deep tumor sites of the lung, resulting insufficient samples for analysis. Therefore, due to the non-invasive and non-radioactive features of screening method using biomarkers, it is crucial for use in clinical diagnosis and treatment.

Current recommended lung cancer biomarkers include carcinoembryonic antigen (CEA), progastrin-releasing peptide (ProGRP), cytokeratin 19 fragment (Cyfra21-1), neuron-specific enolase (NSE), and squamous cell carcinoma (SCC) antigen. However, these markers are unable to determine the exact type of lung cancer. With the availability of personalized targeted therapies, the need for accurate typing of lung cancer becomes crucial. The lung cancer is mainly categorized into two major groups, small cell lung cancer and non-small cell lung cancer. The small cell lung cancer accounts for about 15% and the non-small cell lung cancer accounts for about 85% of all lung cancers. The non-small cell lung cancer is mainly classified into adenocarcinoma and squamous carcinoma. In pathological diagnosis, pathologists usually identify different pathological types of lung cancer using several immunohistochemical staining indexes, such as lung adenocarcinoma biomarkers TTF-1 and Napsin A, squamous cell lung carcinoma biomarkers CK5/6, p63 and p40, and small cell lung cancer biomarkers CgA, SyN and CD56. However, tumors in some patients with lung cancer which are located at special position, such as a mass at the tip of the lung are unable to be sampled clinically for biopsies. In addition, some patients refuse a routine lung mass biopsy due to contraindications for bronchofiberscopy or percutaneous lung puncture biopsy, or due to fear of the risk of invasive biopsy. Therefore, a more comprehensive and safer detection scheme is urgently needed, such as detecting these markers by using peripheral blood to improve the accuracy of diagnosis.

Tumor heterogeneity is an important reason that all molecular information about a tumor is unable to be obtained by a single biopsy and is also a fundamental factor causing drug resistance of the tumor. Hence, dynamic evaluation of molecular classification of tumor is required.

As a non-invasive tumor detection method, liquid biopsy has the advantages of being safe, convenient, sustainable, and dynamic in detection and evaluation. Circulating malignant cells (CMCs) exist in blood of patients with tumors at different development stages from early primary tumors to advanced tumors, including circulating tumor cells (CTCs) and circulating hybrid cells (CHCs). The detection of the CMCs in blood has several advantages compared to the detection of free markers (such as circulating tumor DNA (ctDNA), exosomes and cell-free DNA (cf-DNA)) in the blood. First, the CMCs are able to provide more comprehensive biological information of cancer at a cellular level, which are direct evidence of tumor cell metastasis in vivo that reflect tumor heterogeneity and dynamic changes. The quantity of the CMCs and the heterogeneous state of the markers therein are closely related to the tumor development, and thus are suitable for evaluating prognosis to provide more valuable clinical guidance. However, free markers can only provide single information, and are easily affected by various factors, making it difficult to comprehensively and accurately reflect the real state of the tumor. Therefore, the detection of the CMCs has greater potential and advantages, and has wide application prospects in early screening, curative effect evaluation, prediction of recurrence and metastasis, and the like.

Since the quantity of the CMCs in blood is very small, it is difficult to detect the CMCs in blood in situ. Generally, the CMCs are separated, enriched and then detected. However, methods of first enriching and then characterizing have many disadvantages, for example, damage to the CMCs or change of a cell state can be occurred during the enrichment process, and cell detection after the enrichment excessively depends on various staining methods, resulting in a cumbersome process. In addition, the detection result is likely to be false negative because only some malignant cells are stained with antibodies due to the absence of surface antigens or the heterogeneity of the tumor cells. Therefore, it is particularly important to develop a method that can implement in situ detection of live CMCs to avoid errors caused by the change of the CMCs during the enrichment process, accurately detect malignant cells in the absence of surface antigens, and fully reflect the heterogeneity of the malignant cells. In addition, in the aspect of detection of nucleic acid markers, the current existing gene delivery vectors such as viral vectors and cationic liposome delivery vectors are having the problems of lack of tumor targeting, poor biocompatibility which causes cell oxidative stress reaction easily, and the like, and thus accurate results are unable to be obtained. Therefore, it is urgently needed to develop a delivery vector that has good biocompatibility, does not affect the cell state and has high-efficiency targeting delivery capability for the malignant cells, and able to efficiently deliver a molecular probe for detecting the nucleic acid marker into live malignant cells to realize accurate detection.

### SUMMARY OF THE INVENTION

The present invention aims to provide a method for in situ detection of multiple cancer-related markers in live circulating malignant cells, and tumor heterogeneity. The method realizes the simultaneous detection of a plurality of lung cancer-related nucleic acid markers in live circulating malignant cells in blood and realizes the early diagnosis of lung cancer in situ and the detection of lung cancer heterogeneity at different development stages.

The technical solution utilized in the present invention: a method for in situ detection of multiple lung cancer-related markers in live circulating malignant cells, and tumor heterogeneity, comprising accurately detecting at least one lung cancer-related nucleic acid marker in live lung cancer cells by using a lung cancer-targeted nanoprobe loaded with a plurality of molecular beacons which is configured to reflect levels of different markers in the lung cancer cells from a single cell level and a heterogeneous state of a tumor of a specific patient.

Preferably, the nanoprobe is a nanoparticle self-assembled from a high polymer material, an electropositive protein, a functional polypeptide and/or a functional aptamer, and a molecular beacon of a lung cancer-related marker.

Preferably, the high polymer material comprises at least one of hyaluronic acid, aptamerized hyaluronic acid, polypeptidized hyaluronic acid, carboxymethyl chitosan, aptamerized carboxymethyl chitosan, polypeptidized carboxymethyl chitosan and polylysine.

Preferably, the aptamer is at least one of the following aptamers: aptamers CL4, J18 and Tutu-22 targeting wild-type EGFR (epidermal growth factor receptor), an aptamer ME07 targeting wild-type and mutant lung cancers, an aptamer E21 targeting EGFRIII, aptamers CLN3, CLN4 and CLN64 targeting c-MET (mesenchymal-epithelial transition factor), aptamers SE25-8, HB5 and HY6 targeting HER2 (human epidermal growth factor receptor-2), and an aptamer SYL3C (aptamer against epithelial cell adhesion molecule) targeting EpCAM (epithelial cell adhesion factor), and the polypeptide comprises any one of a TAT penetrating peptide, an NLS nuclear localization peptide, a KALA polypeptide, a peptide GE11 targeting EGFR and a fusion peptide formed through fusion thereof.

Preferably, the electropositive protein comprises any one of protamine, histone and lysozyme.

Preferably, the molecular beacon of a malignant cell-specific related gene comprises at least one of a molecular beacon of a lung cancer marker, a molecular beacon of a lung adenocarcinoma marker, a molecular beacon of a mutant lung cancer marker, a molecular beacon of a squamous cell lung carcinoma marker and a molecular beacon of a small cell lung cancer marker.

Preferably, the molecular beacon of the marker is marked with a fluorophore at a 5′ end and marked with a fluorescence quenching group at a 3′ end; a loop part of the molecular beacon of the marker has 15-30 nts of bases and is used for complementary pairing with a coding sequence (CDS) region of specific RNA of malignant cells; and a stem part of the molecular beacon of the marker has 5-8 nts of bases and is used for forming a hairpin structure that makes the fluorophore and the quenching group close to each other which is configured to quince the fluorescence before the molecular beacon of the marker meets a target. Fluorescence emitted by a fluorophore in the molecular beacon can be quenched by a quenching group. When the molecular beacon binds to a target cell target, a stem loop of the molecular beacon is opened, and the fluorophore is far away from the quenching group, such that a fluorescent signal can be detected.

The CDS region of RNA refers to a moiety of an RNA molecule that can be translated into protein. This region encompasses a sequence from a start codon (usually AUG) to a stop codon (e.g., UAA, UAG or UGA). The CDS region determines the amino acid sequence of the protein and is a critical part of gene expression.

The molecular beacon nanoprobe of the present invention comprises a plurality of specific molecular beacons marked by fluorophores with different colors, and each molecular beacon comprises recognition of one specific target in circulating malignant cells (CMCs). When the molecular beacons do not encounter a target, the fluorophore and the quenching group are close to each other, and at this time, the probe does not fluoresce. When aptamers and targeting polypeptides on the surfaces of nanoparticles are combined with proteins over-expressed on the surfaces of the CMCs, and the nanoparticles enter the CMCs through active targeting endocytosis. The nanoparticles are disintegrated in the CMCs and then release the molecular beacons into cytoplasm. When the molecular beacons hybridize to specific nucleic acid strands containing target sequences in the cytoplasm, they undergo a conformational change. The fluorophores are far from the quenching groups, such that a bright fluorescence is emitted. Since different fluorophores are bonded to different molecular beacons, different targets can be detected in the same cell at the same time. The different types of tumors can be identified through the cells emitting different colors of fluorescence, and the progress of the tumors can be monitored through the strength of the fluorescence and the number of the luminescent cells. The cells in the blood of patients with cancer mainly comprise blood cells and the CMCs. Since red blood cells and platelets have no endocytosis capacity, the nanoparticles cannot enter the red blood cells and the platelets. The surfaces of leukocytes have no protein capable of being specifically combined with the nanoparticles, and the nanoparticles are unable to enter the leukocytes. Therefore, the nanoparticles can efficiently and accurately detect the CMCs in blood and discriminate different types of the CMCs by different fluorescence emission so as to distinguish different types of tumors.

Preferably, the nucleic acid detectable by the molecular beacon nanoprobe comprises any three of general tumor markers Ki67 mRNA (5′-CAAGGCACCAAAGAGUGAGAAAGG-3′; SEQ ID NO: 1), c-Myc mRNA (5′-CGAGACCUUCAUCAAAAACAUCAUCAUCCA-3′; SEQ ID NO: 2), lung cancer markers EGFR mRNA (5′-CAACGAAUGGGCCUAAGAUCCCGUCC-3′; SEQ ID NO: 3), CEA mRNA, ProGRP mRNA, Cyfra21-1 mRNA, NSE mRNA, and SCC mRNA, a mutant lung cancer marker EGFR E746_A750del mRNA (5′-UCGCUAUCAAGACAUCUCCGAAAGCC-3′; SEQ ID NO: 4), lung adenocarcinoma markers TTF-1 mRNA (5′-UCGGGAAAAACUCUACAAGGGCAUA-3′; SEQ ID NO: 5), NapsinA mRNA (5′-UCCAGACUACUCGAAAUGGCGUCC-3′; SEQ ID NO: 6) and CK7 mRNA, carcinoma markers CK5/6 mRNA (5′-squamous cell lung GUUCGAGCAGUACAUCAACAACCUCAGG-3′; SEQ ID NO: 7), p63 mRNA (5′-GAAGAGACAGGAAGGCGGAUGAAGAUAGCA-3′; SEQ ID NO: 8) and p40 mRNA, and small cell lung cancer markers CgA mRNA, SyN mRNA (5′-CAACCUGUGGUUCGUGUUUAAGGAGACAG-3′; SEQ ID NO: 9) and CD56 mRNA (5′-GAGUGGAGAGCAGUUGGUGAAGAAGUAUGG-3′; SEQ ID NO: 10).

The general tumor marker molecular beacon is at least one of molecular beacons for detecting Ki67 and c-Myc genes, the lung cancer marker molecular beacon is at least one of molecular beacons for detecting EGFR, CEA, ProGRP, Cyfra21-1, NSE and SCC genes, the molecular beacon of a mutant lung cancer marker is at least one of molecular beacons for detecting specific mutant lung cancer gene EGFR E746_A750del, the molecular beacon of a lung adenocarcinoma marker is at least one of molecular beacons for detecting TTF-1, NapsinA and CK7 genes, the molecular beacon of a squamous cell lung carcinoma marker is at least one of molecular beacons for detecting CK5/6, p63 and p40 genes, and the molecular beacon of a small cell lung cancer marker is at least one of molecular beacons for detecting CgA, SyN and CD56 genes.

Preferably, a method for preparing the nanoprobe comprises the following steps:
(1) adding deionized water into specific amounts of the electropositive protein and the functional polypeptide and/or the functional aptamer to prepare a solution A, adding deionized water into a specific amount of a solution of the molecular beacon of the lung cancer-related marker to prepare a solution B, dropwise adding the solution A into the solution B, and uniformly mixing same to obtain a mixed solution; and
(2) adding the high polymer material into the mixed solution obtained in step (1) and continuously and uniformly mixing same to obtain the nanoprobe.

Synthesis principle: the nanoprobe is synthesized by a self-assembly method. Protamine@functional polypeptide/molecular beacon, histone@functional polypeptide/molecular beacon, or lysozyme@functional polypeptide/molecular beacon nanoparticles are formed by using positively charged protamine sulfate, histone or lysozyme, functional polypeptides with negatively charged molecular beacons through electrostatic interaction. Then, a negatively charged high polymer material is added into the protamine@functional polypeptide/molecular beacon, histone@functional polypeptide/molecular beacon or lysozyme@functional polypeptide/molecular beacon nanoparticles with positively charged surfaces, thereby immediately preparing a biological high polymer material protamine @functional polypeptide/molecular beacon, histone@functional polypeptide/molecular beacon or lysozyme@functional polypeptide/molecular beacon nanoprobe.

Preferably, when the high polymer material is selected from hyaluronic acid, carboxymethyl chitosan, sodium alginate, and heparin sodium, the high polymer material is firstly subjected to aptamatization by step I or II or subjected to polypeptidization by step III or IV.
I. A high polymer material containing carboxyl is dissolved in a PBS (pH=6) solution, then a catalyst EDC/NHS is added for activation at room temperature, then an aminated aptamer or polypeptide is added, and reaction is performed at room temperature; and the product obtained after the reaction is put into a dialysis bag for dialysis and freeze-dried to obtain the functional high polymer material.
II. The carboxylated aptamer or polypeptide is dissolved in a PBS buffer solution and activated by using the catalyst EDC/NHS at room temperature, a high polymer material is added, and reaction is performed at room temperature; and the product obtained after the reaction is put into the dialysis bag for dialysis and freeze-dried to obtain the functional high polymer material.

In steps I and II, after the catalyst EDC/NHS is added, the molar ratio of —COOH, EDC and NHS in the solution is 1:1.2:1.2, and the molar ratio of the high polymer material to the aptamer or the polypeptide is 10:1.

When the high polymer material is hyaluronic acid, sodium alginate and heparin sodium, the functional high polymer material is prepared by using method I. When the high polymer material is carboxymethyl chitosan, the functional high polymer material is prepared by using method II.

Preferably, the nanoprobe can be used in the preparation of a detection product of lung nodules, lung adenocarcinoma, squamous cell lung carcinoma, and small cell lung cancer.

Preferably, the nanoprobe can be used to simultaneously detect a plurality of nucleic acid markers and reflect the levels of different markers in the malignant cells from a single cell level, thereby reflecting the tumor heterogeneous state of the specific patient.

The present invention has the following advantages and beneficial effects:

The tumor targeting nanoprobe of the present invention loaded with a plurality of molecular beacons accurately detects nucleic acid markers in live malignant cells.

The nanoprobe of the present invention can be used to target all circulating malignant cells in whole blood, simultaneously detect a plurality of nucleic acid markers in living cells, which is configured to reflect the levels of different markers in the malignant cells from a single cell level, and thereby reflecting the tumor heterogeneous state of the specific patient and realizing early accurate diagnosis of lung cancer.

All materials of the molecular beacon nanoprobe of the present invention are biocompatible materials, such that toxic and side effects of blood cells and circulating malignant cells will not be caused by direct incubation in blood, and the detection of target genes in the malignant cells will not be interfered. The particle sizes and the potentials of the nanoparticles of the molecular beacon nanoprobe of the present invention are able to meet the requirements of entering cells, and meanwhile, the nanoparticles have good stability and biocompatibility.

A preparation principle of the molecular beacon nanoprobe of the present invention belongs to electrostatic interaction. Besides, all the processes are performed in a water phase, the preparation process is simple and efficient, and different targeting aptamers and functional polypeptide molecules can be bonded to high polymer chains on the surfaces of the synthesized nanoparticles, such that the nanoparticles can efficiently reach malignant cell parts in a targeted manner in a complex blood environment. The molecular beacons can be wrapped in the nanomaterial to protect the beacons from enzymatic degradation in blood and no immunogenic reaction is caused. Meanwhile, after the nanoparticles enter the circulating malignant cells, the nanoparticles are disintegrated in the circulating malignant cells, and the molecular beacons are efficiently released into cytoplasm, such that the beacons are efficiently combined with target nucleic acid sequences, a bright fluorescence is emitted, and the purpose of identifying target cells is fulfilled.

The preparation method of the present invention is non-toxic, simple and quick synthesis process, in which the whole process is performed in a water phase, and mass production can be performed.

The nanoprobe loaded with molecular beacons of the present invention is directly added into a blood sample of a patient with cancer, the nanoprobe transmits the molecular beacons to malignant cells in whole blood in a targeted manner, and a plurality of the molecular beacons are respectively hybridized with different nucleic acid molecules in the malignant cells in a living cell state. Before detection, separation and enrichment of the circulating malignant cells are not required, such that the damage to the cells caused by the cell enrichment and cell detection is avoided. The molecular beacon nanoprobe of the present invention can be used for detecting lung cancer-related nucleic acid markers in a plurality of the circulating malignant cells simultaneously, and the molecular beacons and the nucleic acid markers are hybridized to emit fluorescence of different colors, such that the molecular beacon nanoprobe can be used for early detection of lung cancer and determination of accurate classification of the lung cancer. Particularly, for early stage lung tumor, the detection offers accurate diagnosis of benign and malignant tumors and accurate categorization of the lung cancer, and thus effectively helps the customization of a personalized treatment scheme.

The molecular beacon nanoprobe of the present invention can promote cell specific targeting and uptake, and endosome escape by adding the functional polypeptides, such that the functional polypeptides are introduced into the core and/or the surface of the nanoprobe so as to further improve the delivery efficiency of the molecular beacons.

### BRIEF DESCRIPTION OF THE DRAWINGS

FIG. 1 is a structure diagram of a molecular beacon (MB);

FIG. 2 is a schematic diagram of an action principle of a nanoprobe of the present invention;

FIG. 3 explores the sensitivity of detection of the molecular beacons;

FIG. 4 shows a synthesis method of an aptamerized high polymer in the molecular beacon nanoprobe of the present invention and the composition of a functional polypeptide;

FIG. 5 is the characterization of the nanoprobe of the present invention;

FIG. 6 is a diagram showing the detection of nanoprobe in Example 4 of the present invention indicating the expression levels of different tumor markers in representative CMCs in 2 ml of whole blood of a real patient;

FIG. 7 is a diagram showing the detection of nanoprobe in Example 4 of the present invention indicating the expression levels of different tumor markers in all CMCs in 2 ml of whole blood of a real patient; and

FIG. 8 is the basis of pathological diagnosis.

### DETAILED DESCRIPTION OF THE EMBODIMENTS

To better understand the present invention, the following examples further illustrate the present invention, but the content of the present invention is not limited to the following examples.

### Example 1 Design and Optimization of Probe

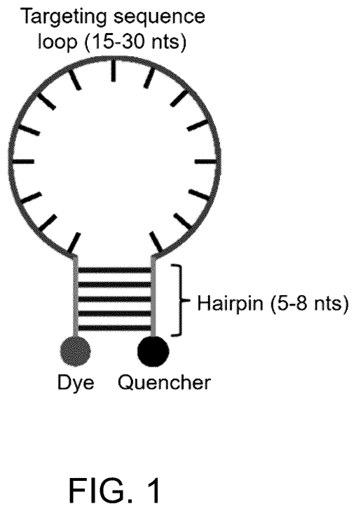

Based on literature research and through a molecular beacon design tool (Beacon designer), molecular beacons for detecting target genes Ki67 mRNA, c-Myc mRNA and TTF1 mRNA were constructed. The structure of the molecular beacon is shown in FIG. 1. A sequence of the molecular beacon for detecting Ki67 mRNA (abbreviated as MB_{Ki67}) is 5′-Alexa fluor 488-CGACCCTTTCTCACTCTTTGGTGCCTTGGTCG-Dabcyl-3′ (SEQ ID NO: 11). A sequence of the molecular beacon for detecting c-Myc mRNA (abbreviated as MB_{c-Myc}) is 5′-Cy5-CACTGTGGATGATGATGTTTTTGATGAAGGTCTCGCAGTG-Dabcyl-3′ (SEQ ID NO: 12). A sequence of the molecular beacon for detecting TTF1 mRNA (abbreviated as MB_{TTF1}) is 5′-Cy3-ACCGTATGCCCTTGTAGAGTTTTTCCCGACGGT-Dabcyl-3′ (SEQ ID NO: 13). All the designed MBs were synthesized by Sangon Biotech (Shanghai) Co., Ltd. These molecular beacons were optimized based on actual detection effects.

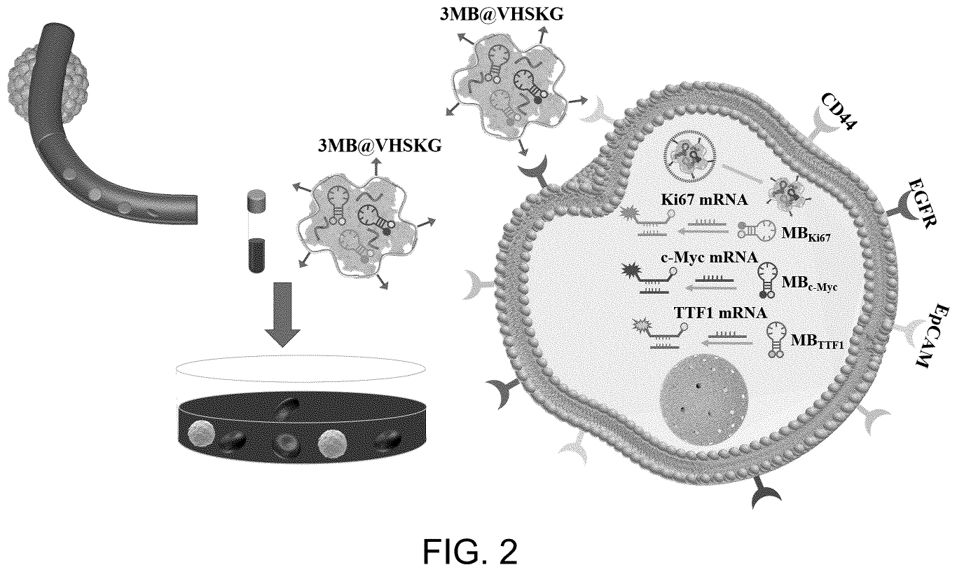

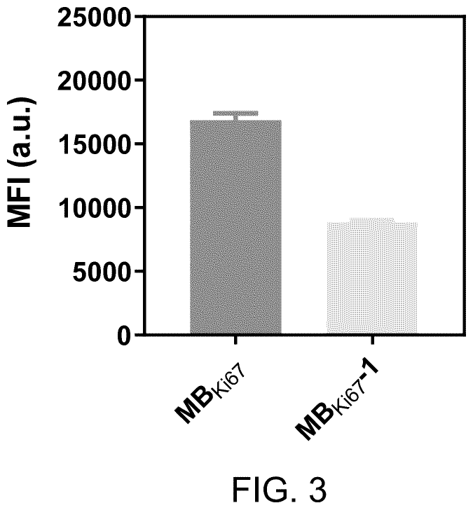

To investigate the sensitivity of the molecular beacon detection, MB_{Ki67} (0.07 nmol, dissolved in 7 μL of ultrapure water) and MB_{Ki67-1} (0.07 nmol, dissolved in 7 μL of ultrapure water) were respectively incubated with a commercial carrier Lip2000 (1 μg, dissolved in 93 μL of ultrapure water) for 15 minutes to obtain nanoprobes MB_{Ki67}@Lip and MB_{Ki67}-1@Lip. Cells were inoculated in a 6-well plate (2×10⁵ cells in 2 mL of medium per well). After the incubation for 24 h, the medium was replaced respectively with 1 mL of fresh medium containing 3MB_{Ki67}@Lip and 1 mL of fresh medium containing MB_{Ki67}-1@Lip. After the incubation at 37° C. for 10 h, the obtained product was washed with PBS, digested with trypsin, centrifuged and collected, fixed with 4% paraformaldehyde, filtered, and analyzed with a flow cytometer (Dakewe Biotech EXFLOW 206). It can be learned from FIG. 3 that the fluorescence intensity of MB_{Ki67}-1@Lip is significantly lower than that of MB_{Ki67}@Lip, indicating that the sequence of the molecular beacon MBKi67 has higher detection sensitivity. The schematic diagram of the function of the nanoprobe of the present invention is shown in FIG. 2.

### Example 2 Preparation of Nanoprobe

### 1. Synthesis of SYL3C Binding to HA (SHA)

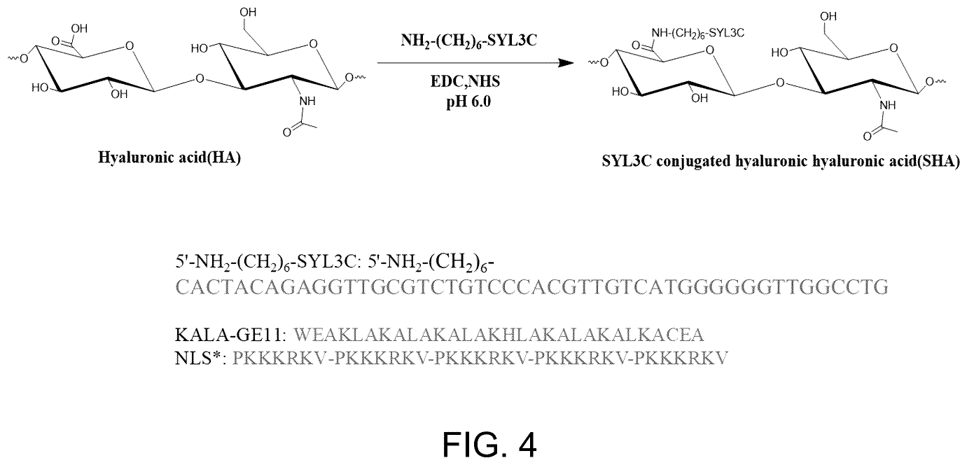

Hyaluronic acid (304 μg) was dissolved in a PBS buffer (pH=6.0, 1 mL), activated with a catalyst (EDC/NHS) (the molar ratio of —COOH:EDC:HOBt=1:1.2:1.2) at room temperature for 1 hour, an aptamer SYL3C (586 μg) (CACTACAGAGGTTGCGTCTGTCCCACGTTGTCATGGGGGGTTGGCCTG; SEQ ID NO: 14) was added, and the reaction was performed at room temperature for 24 hours. After reacting with the high polymer material, the obtained product was put into a dialysis bag (MWCO 20000) for dialyzing in ultrapure water for 72 hours to remove unreacted aptamer and other impurities, and then the product was freeze-dried to obtain SHA. FIG. 4 shows a synthesis method of an aptamerized high polymer in the molecular beacon nanoprobe of the present invention and the composition of a functional polypeptide.

2. Synthesis of Nanoprobe Loaded with MB_{Ki67}, MB_{c-Myc} and MB_{TTF1}

MB_{Ki67} (0.07 nmol dissolved in 7 μL of ultrapure water), MB_{c-Myc} (0.07 nmol dissolved in 7 μL of ultrapure water) and MB_{TTF1} (0.07 nmol dissolved in 7 μL of ultrapure water) were mixed to form a mixed solution A, histone (30 μg) and NLS* (3 μg) (PKKKRKVPKKKRKVPKKKRKVPKKKRKVPKKKRKV; SEQ ID NO: 15) were mixed in ultrapure water (36 μL) to form a mixed solution B, the mixed solution A was added dropwise to the mixed solution B, and after gently mixing for 15 minutes, an electropositive MBₖᵢ₆₇/MB_{c-Myc}/MB_{TTF1}@Histone/NLS* mixed solution was obtained. Then 43 μL of the electronegative mixed solution containing SHA (6 μg) and KALA-GE11 (1 μg) (WEAKLAKALAKALAKHLAKAKALKACEA; SEQ ID NO: 16) was added dropwise to the MBₖᵢ₆₇/MB_{c-Myc}/MB_{TTF1}@Histone/NLS* mixed solution, and after gently mixing for 15 minutes, a triple targeting nanoprobe (MBₖᵢ₆₇/MB_{c-Myc}/MB_{TTF1}@Histone/NLS*/SHA/KALA-GE11 abbreviated as “3MB@VHSKG”) was obtained.

### Example 3 Characterization of Nanoprobe

### 1. Determination of Particle Size, Potential and Encapsulation Efficiency of 3MB@VHSKG Nanoparticle

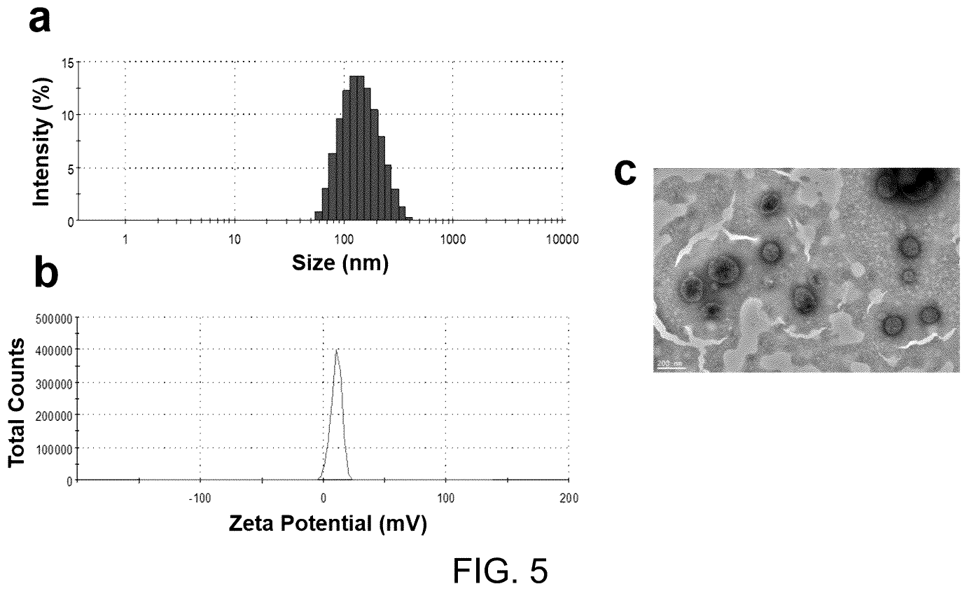

The 3 MB@VHSKG nanoparticle solution prepared in example 2 was diluted with deionized water to a total volume of 1 mL. The size and potential of the 3 MB@VHSKG nanoparticle in deionized water were measured by Zetasizer (Nano ZS, Malvern Instruments). Experimental results are shown in a and b of FIG. 5. It can be seen that the hydrodynamic size of the nanoparticle is less than 300 nm, the potential is greater than 0, and the nanoparticle is suitable for cellular uptake.

To determine the encapsulation efficiency of molecular beacons, the 3 MB@VHSKG nanoparticle solution was centrifuged (10,000 rpm) at 4° C. for 1 hour at a specified rotation speed, then the amount of non-precipitated free molecular beacons remaining in the supernatant was determined, and the encapsulation efficiency of the molecular beacons was calculated as the ratio of the amount of precipitated beacons to the total input amount. The results of the experiment indicate that the encapsulation efficiency of the molecular beacons is ninety percent or greater.

### 2. Morphology of 3 MB@VHSKG Nanoparticle Under Transmission Electron Microscope (TEM)

An ultrathin carbon support film was infiltrated by using a sample solution, a small amount of a phosphotungstic acid solution (0.001 mol/L) was added for infiltration negative dyeing after the 3 MB@VHSKG nanoparticle was deposited, and the sample was volatilized and air-dried at room temperature. Finally, the sample was observed by a transmission electron microscope (JEM-2100). The experimental results are shown in c of FIG. 5. It can be known from the figure that the 3 MB@VHSKG nanoparticle shows a uniformly dispersed spherical shape.

### Example 4 Detecting Nucleic Acids in CMCs in Blood Samples of Patient with Tumor by Using 3 MB@VHSKG

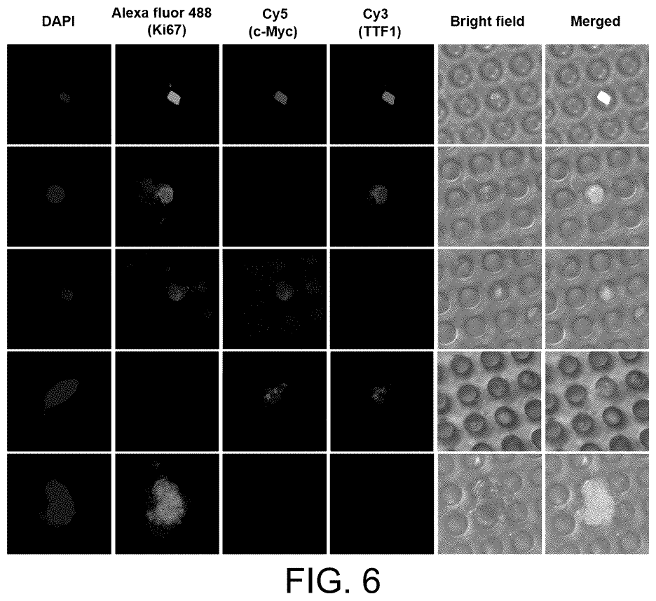

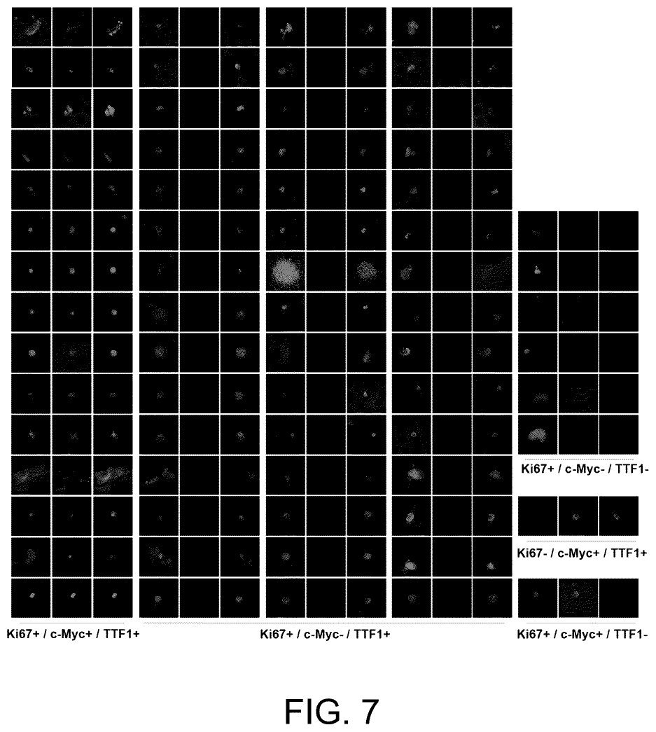

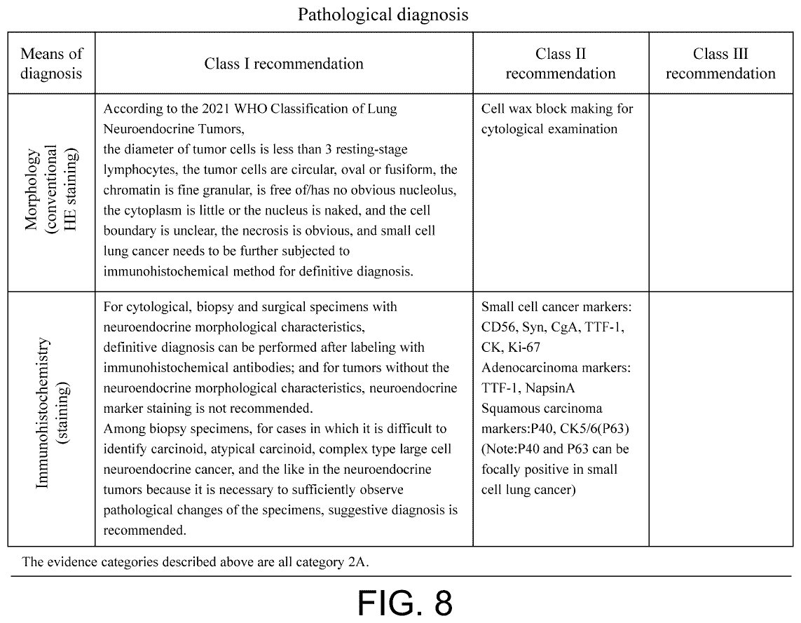

2 ml of blood of a patient with early lung nodule was taken in an EDTA anticoagulation tube, then the 3 MB@VHSKG nanoparticle solution was added into the blood, after incubation for 10 hours, the blood was placed on a filtering membrane with the diameter of 7 microns for filtering, the filtering membrane was collected in a confocal cuvette, cell nuclei (blue) were stained by DAPI, and then the cells were observed by a laser confocal microscope. The experimental results are shown in FIG. 6 and FIG. 7. The fluorescence intensity and the type of fluorescence emitted by each circulating tumor cell are different, indicating that the tumor cells had heterogeneity. The circulating tumor cells emitting a green fluorescence indicate that the tumor cells are Ki67 positive, the lung nodule is malignant. Most circulating tumor cells emitting a rose red fluorescence indicate that the tumor cells are one of lung adenocarcinoma or small cell lung cancer, and the possibility of squamous cell lung carcinoma is excluded. The results can provide reference for the diagnosis of benign and malignant early lung nodule and the accurate diagnosis of lung cancer types. FIG. 8 is the basis of pathological diagnosis.

The above descriptions are merely preferred implementations of the present invention and certainly cannot be used to define the claims of the present invention. It should be noted that a person of ordinary skill in the art may further make several improvements and modifications without departing from the principle of the present invention, but such improvements and modifications should be deemed as falling within the protection scope of the present invention.

What is claimed is:

1. A method for in situ detection of multiple lung cancer-related markers in live circulating malignant cells, and tumor heterogeneity, comprising
accurately detecting at least one lung cancer-related nucleic acid marker in live lung cancer cells by using a lung cancer-targeted nanoprobe loaded with a plurality of molecular beacons which is configured to reflect levels of different markers in the lung cancer cells from a single cell level and a heterogeneous state of a tumor of a specific patient.

2. The method for in situ detection of the multiple lung cancer-related markers in the live circulating malignant cells, and the tumor heterogeneity according to claim 1, wherein the lung cancer-targeted nanoprobe is a nanoparticle self-assembled from a high polymer material, an electropositive protein, a functional polypeptide and/or a functional aptamer, and a molecular beacon of a lung cancer-related marker.

3. The method for in situ detection of the multiple lung cancer-related markers in the live circulating malignant cells, and the tumor heterogeneity according to claim 2, wherein the high polymer material comprises at least one of hyaluronic acid, aptamerized hyaluronic acid, polypeptidized hyaluronic acid, carboxymethyl chitosan, aptamerized carboxymethyl chitosan, polypeptidized carboxymethyl chitosan and polylysine.

4. The method for in situ detection of the multiple lung cancer-related markers in the live circulating malignant cells, and the tumor heterogeneity according to claim 3, wherein an aptamer of the aptamerized hyaluronic acid and the aptamerized carboxymethyl chitosan is at least one of the following aptamers: aptamers CL4, J18 and Tutu-22 targeting wild-type EGFR, an aptamer ME07 targeting wild-type and mutant lung cancers, an aptamer E21 targeting EGFRIII, aptamers CLN3, CLN4 and CLN64 targeting c-MET, aptamers SE25-8, HB5 and HY6 targeting HER2, and an aptamer SYL3C targeting EpCAM, and a polypeptide of the polypeptidized hyaluronic acid and the polypeptidized carboxymethyl chitosan comprises any one of a TAT penetrating peptide, an NLS nuclear localization peptide, a KALA polypeptide, a peptide GE11 targeting EGFR and a fusion peptide formed through fusion thereof.

5. The method for in situ detection of the multiple lung cancer-related markers in the live circulating malignant cells, and the tumor heterogeneity according to claim 2, wherein the electropositive protein comprises any one of protamine, histone and lysozyme.

6. The method for in situ detection of the multiple lung cancer-related markers in the live circulating malignant cells, and the tumor heterogeneity according to claim 2, wherein the molecular beacon of the lung cancer-related marker comprises at least one of a molecular beacon of a lung cancer marker, a molecular beacon of a lung adenocarcinoma marker, a molecular beacon of a mutant lung cancer marker, a molecular beacon of a squamous cell lung carcinoma marker and a molecular beacon of a small cell lung cancer marker.

7. The method for in situ detection of the multiple lung cancer-related markers in the live circulating malignant cells, and the tumor heterogeneity according to claim 6, wherein the molecular beacon of the marker is marked with a fluorophore at a 5′ end and marked with a fluorescence quenching group at a 3′ end; a loop part of the molecular beacon of the marker has 15-30 nts of bases and is used for complementary pairing with a coding sequence (CDS) region of malignant cell-specific RNA; and a stem part of the molecular beacon of the marker has 5-8 nts of bases and is used for forming a hairpin structure that makes the fluorophore and the fluorescence quenching group close to each other which is configured to quench a fluorescence of the fluorophore before the molecular beacon of the marker meets a target of the bases.

8. The method for in situ detection of the multiple lung cancer-related markers in the live circulating malignant cells, and the tumor heterogeneity according to claim 2, wherein a nucleic acid detectable by the lung cancer-targeted nanoprobe comprises at least one of general tumor markers Ki67 mRNA and c-Myc mRNA, lung cancer markers EGFR mRNA, CEA mRNA, ProGRP mRNA, Cyfra21-1 mRNA, NSE mRNA and SCC mRNA, a mutant lung cancer marker EGFR E746_A750del mRNA, lung adenocarcinoma markers TTF-1 mRNA, NapsinA mRNA and CK7 mRNA, squamous cell lung carcinoma markers CK5/6 mRNA, p63 mRNA and p40 mRNA, and small cell lung cancer markers CgA mRNA, SyN mRNA and CD56 mRNA.

9. The method for in situ detection of the multiple lung cancer-related markers in the live circulating malignant cells, and the tumor heterogeneity according to claim 2, wherein a method for preparing the lung cancer-targeted nanoprobe comprises the following steps:
(1) adding deionized water into specific amounts of the electropositive protein and the functional polypeptide and/or the functional aptamer to prepare a solution A, adding deionized water into a specific amount of a solution of the molecular beacon of the lung cancer-related marker to prepare a solution B, dropwise adding the solution A into the solution B, and uniformly mixing same to obtain a mixed solution; and
(2) adding the high polymer material into the mixed solution obtained in step (1) and continuously and uniformly mixing same to obtain the lung cancer-targeted nanoprobe.

10. The method for in situ detection of the multiple lung cancer-related markers in the live circulating malignant cells, and the tumor heterogeneity according to claim 2, wherein the lung cancer-targeted nanoprobe is used in preparation of a detection product of lung nodules, lung adenocarcinoma, squamous cell lung carcinoma, and small cell lung cancer.


SEQUENCE LISTING (16 sequences)

SEQ ID NO: 1 - RNA, 24 nt, synthetic construct
  source 1..24: mol_type = other RNA
caaggcaccaaagagtgagaaagg

SEQ ID NO: 2 - RNA, 30 nt, synthetic construct
  source 1..30: mol_type = other RNA
cgagaccttcatcaaaaacatcatcatcca

SEQ ID NO: 3 - RNA, 26 nt, synthetic construct
  source 1..26: mol_type = other RNA
caacgaatgggcctaagatcccgtcc

SEQ ID NO: 4 - RNA, 26 nt, synthetic construct
  source 1..26: mol_type = other RNA
tcgctatcaagacatctccgaaagcc

SEQ ID NO: 5 - RNA, 25 nt, synthetic construct
  source 1..25: mol_type = other RNA
tcgggaaaaactctacaagggcata

SEQ ID NO: 6 - RNA, 24 nt, synthetic construct
  source 1..24: mol_type = other RNA
tccagactactcgaaatggcgtcc

SEQ ID NO: 7 - RNA, 28 nt, synthetic construct
  source 1..28: mol_type = other RNA
gttcgagcagtacatcaacaacctcagg

SEQ ID NO: 8 - RNA, 30 nt, synthetic construct
  source 1..30: mol_type = other RNA
gaagagacaggaaggcggatgaagatagca

SEQ ID NO: 9 - RNA, 29 nt, synthetic construct
  source 1..29: mol_type = other RNA
caacctgtggttcgtgtttaaggagacag

SEQ ID NO: 10 - RNA, 30 nt, synthetic construct
  source 1..30: mol_type = other RNA
gagtggagagcagttggtgaagaagtatgg

SEQ ID NO: 11 - DNA, 32 bp, synthetic construct
  source 1..32: mol_type = other DNA
cgaccctttctcactctttggtgccttggtcg

SEQ ID NO: 12 - DNA, 40 bp, synthetic construct
  source 1..40: mol_type = other DNA
cactgtggatgatgatgtttttgatgaaggtctcgcagtg

SEQ ID NO: 13 - DNA, 33 bp, synthetic construct
  source 1..33: mol_type = other DNA
accgtatgcccttgtagagtttttcccgacggt

SEQ ID NO: 14 - DNA, 48 bp, synthetic construct
  source 1..48: mol_type = other DNA
cactacagaggttgcgtctgtcccacgttgtcatggggggttggcctg

SEQ ID NO: 15 - protein, 35 aa, synthetic construct
PKKKRKVPKKKRKVPKKKRKVPKKKRKVPKKKRKV

SEQ ID NO: 16 - protein, 28 aa, synthetic construct
WEAKLAKALAKALAKHLAKAKALKACEA